(19) United States
(12) Patent Application Publication
(10) Pub. No.: US 2009/0264680 A1
(43) Pub. Date: Oct. 22, 2009

(54) Process for the synthesis and purification of (4-methoxybutyl) (4-trifluoromethylphenyl) methanone

(76) Inventors: Andrea Castellin, Mestrino (IT); Luca Gregori, Padova (IT); Guadalupe Gonzales Trueba, Padova (IT); Albino Rubello, Rovigo (IT); Andrea Nicole, Padova (IT)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 11/630,866

(22) PCT Filed: Jul. 7, 2004
(86) PCT No.: PCT/EP04/51390

§ 371 Date: Oct. 22, 2008

(51) Int. Cl. C07C 45/00

(52) U.S. Cl. 568/322

(57) ABSTRACT

A process for the preparation of 4-trifluoromethylvalerophenone is described. The process described is a three step process comprising the synthesis of organomagnesium specie, coupling reaction between the organomagnesium specie and trifluoromethylbenzonitrile or trifluoromethylbenzoyl chloride and preferably a purification of the product obtained in suitable reaction conditions. In the process an extraction phase of the final product is not required.

### FIELD OF THE INVENTION

The present invention relates to a process for the preparation of 4-trifluoromethylvalerophenone ([5-methoxy-1-(4-trifluoromethyl-phenyl)-pentan-1-one] and its purification.

### PRIOR ART

The compound 4-trifluoromethylvalerophenone ([5-methoxy-1-(4-trifluoromethyl-phenyl)-pentan-1-one] is the starting material for the synthesis of fluvoxamine maleate, important active principle in antidepressant drugs of the class of serotonin uptake inhibitors.

The formation of ketons by addition of Grignard reagents to nitriles and subsequent hydrolysis, is a common preparation known by a person skilled in the art (Karasch, Reinmuth Grignard reactions of non-metallic substances, Prentice-Hall: Englewood Cliffs, N.J., 1954, pp 767-845) Example of synthetic use of 4-methoxybutylmagnesium chloride or the corresponding bromide with electrophilic species are reported: by Curtois et al. in Bull. Soc. Chim. Fr., 2, 5-6; 1983; 148-152; by Cuvigny et coll. In Bull. Soc. Chim. Fr., 1960, 515-521; and by Trahanovsky in J. Am. Chem. Soc.; 96; 1974; 7968-7974.

With reference to 4-trifluoromethylvalerophenone, a process to prepare this intermediate has been described in WO9958485 directed to the synthesis of (alkoxyalkyl) (4-trifluoromethylphenyl)methanones. The features of the process described are essentially pertaining to the conditions of the reaction of 4-trifluoromethylbenzonitrile with an alkoxyalkyl Grignard RO(CH₂)ₙM(X), where X is an halogen and preferentially Br in polar aprotic solvents. The alkoxyalkyl Grignard RO(CH₂)ₙM(X) were obtained reacting the corresponding halide, preferentially bromide, with magnesium in polar aprotic solvents in an inert atmosphere (i.e. dry nitrogen atmosphere). In particular it is relevant for both these reactions the employment of suitable polar aprotic solvents. The polar aprotic solvents are: tetrahydrofuran, diisopropyl ether, dietyl ether, t-butylmethyl ether, 1,2-dimethoxyethane and mixture of the same, being the preferred tetrahydrofuran, diisopropyl ether, dietyl ether, t-butylmethyl ether and tetrahydrofuran the most preferred. The reaction is carried out preferably adding the trifluorobenzonitrile to a suitable alkoxyalkyl Grignard reagent at temperatures comprised from −40° C. to about reflux temperature of the solvent and preferably between 10° C. to 20° C. for a period of about 30 minutes to about 10 hours and preferentially from 1 hour to about 3-4 hours. The reaction mixture is worked up by quenching with saturated ammonium chloride or by adding hydrochloric acid solution and the aqueous layer is further extracted with dichloromethane after separation of organic layer. After extraction with dichloromethane the organic extracts are dried and then the obtained product may be further purified by crystallisation or distillation or by column chromatography. In the example given the yield for the product is 71.84% and the 4-trifluoromethylvalerophenone is purified by distillation. Analytical and structural data for the product are given: melting point 40-42° C. and ¹H NMR, but not purity data of the product are mentioned.

The process described presents some important disadvantages: i) the use of polar aprotic in the both reactions phases resulting in a tedious solvent exchange in the phase of separation of the final product being the solvents, and in particular tetrahydrofuran, miscible with water; ii) the use of dichloromethane in the separation phase with the well known environmental impact typical for chlorinated solvents; iii) further purification after dryness of the dichloromethane extracts. Nevertheless the choice of the polar aprotic solvents, and in particular of tetrahydrofuran, results to be obliged, being these solvents, as well known, the suitable reaction media for Grignard reactions. Actually the solvents and the other conditions of reaction are crucial in influencing the formation of reactive organomagnesium species and even slight changes in these conditions, have a wide effect on the resulting organomagnesium specie output.

In order to implement an efficient industrial process for the preparation of 4-trifluoromethylvalerophenone one purpose is to improve the efficiency of the specific organomagnesium specie synthesis and a second purpose is to avoid the step of separation of the final product with chlorinated solvents. The technical problem to be solved with reference to these purposes refers essentially in finding out appropriate reaction conditions either for the Grignard reaction and for separation of the final product avoiding extraction of the same.

### SUMMARY

According to these purposes for an efficient, cost effective and with low environmental impact industrial process for the synthesis and purification of 4-trifluoromethylvalerophenone, particular attention has to be given to the reagents and in particular to the solvents employed as reaction medium, notwithstanding the technical features mentioned above for the synthesis of organomagnesium species. Furthermore in the light to the economic value of this intermediate for its use for the synthesis of fluvoxamine, the purity of the final products is of great importance.

At these aims the Applicant has developed a new process for the synthesis and purification of 4-trifluoromethylvalerophenone, the essential features of which are different reaction conditions either for Grignard reaction and coupling reaction, in spite the well known technical problems connected with the synthesis of organomagnesium species.

The preparation of the suitable organomagnesium specie is in fact the key point in the process of 4-trifluoromethylvalerophenone synthesis. The solvents employed in the Grignard reaction step in fact strongly influence the separation of the final product and its purification after the coupling reaction.

The Applicant has surprisingly found that changing the reaction conditions for Grignard and coupling reactions fulfils the above-mentioned purposes, leading to a significant improvement of the process, combined with a recovery of the compound 4-trifluoromethylvalerophenone of high purity.

Therefore it is an object of the present invention a process for the synthesis of 4-trifluoromethylvalerophenone characterised by the following steps and conditions:
synthesis of organomagnesium specie CH₃O(CH₂)₄MgX, where X is an halogen, reacting CH₃O(CH₂)₄X with Mg in presence of a suitable initiator in a reaction medium formed by organic solvents selected in the group consisting of 2-methyl-tetrahydrofuran and mixtures thereof with polar aprotic ethereal solvents or apolar aprotic non chlorinated solvents;
synthesis and recovery of 4-trifluoromethylvalerophenone by a coupling reaction of CH₃O(CH₂)₄MgX by adding in the reaction mixture first obtained 4-trifluoromethylbenzonitrile or 4-trifluoromethylbenzoyl chloride in the same organic solvents of the first step, treating the mixture reaction obtained with aqueous acidic diluted solutions, separating the organic layer and concentrating the same to dryness.

A further step of purification is preferably added, said purification being performed at the following conditions:
purification of 4-trifluoromethylvalerophenone obtained by distillation or crystallisation from solutions of the crude product with solvents having boiling point not less than 35° C. and below 145° C.

### BRIEF DESCRIPTION OF THE DRAWINGS

FIG. 1: Analytical Profile of the final product 4-trifluoromethylvalerophenone—¹H-NMR (CDCl₃ 200 MHz) spectra

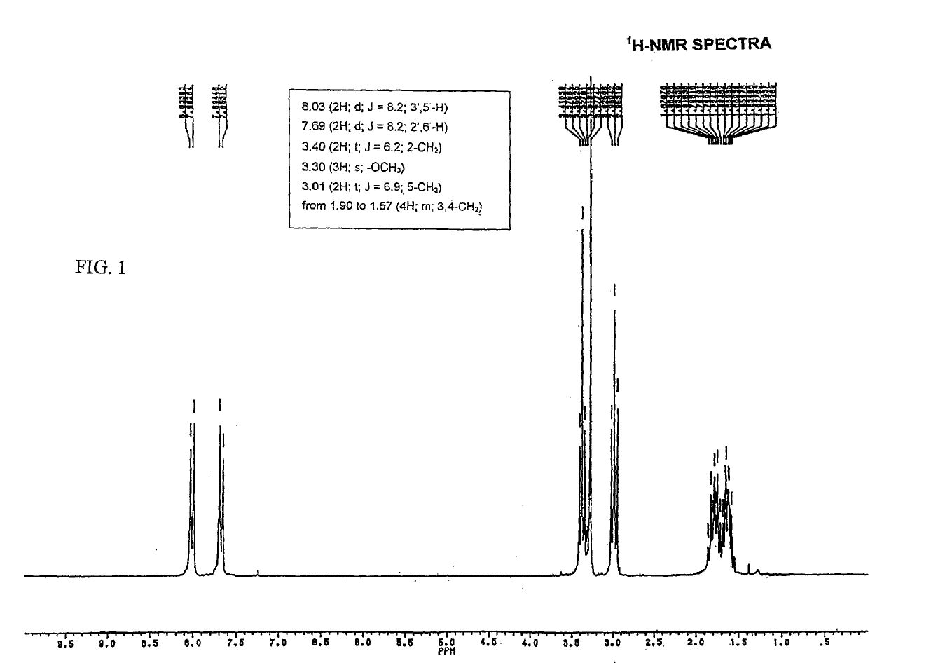

FIG. 2: Analytical Profile of the final product 4-trifluoromethylvalerophenone—IR spectra of the isolated product

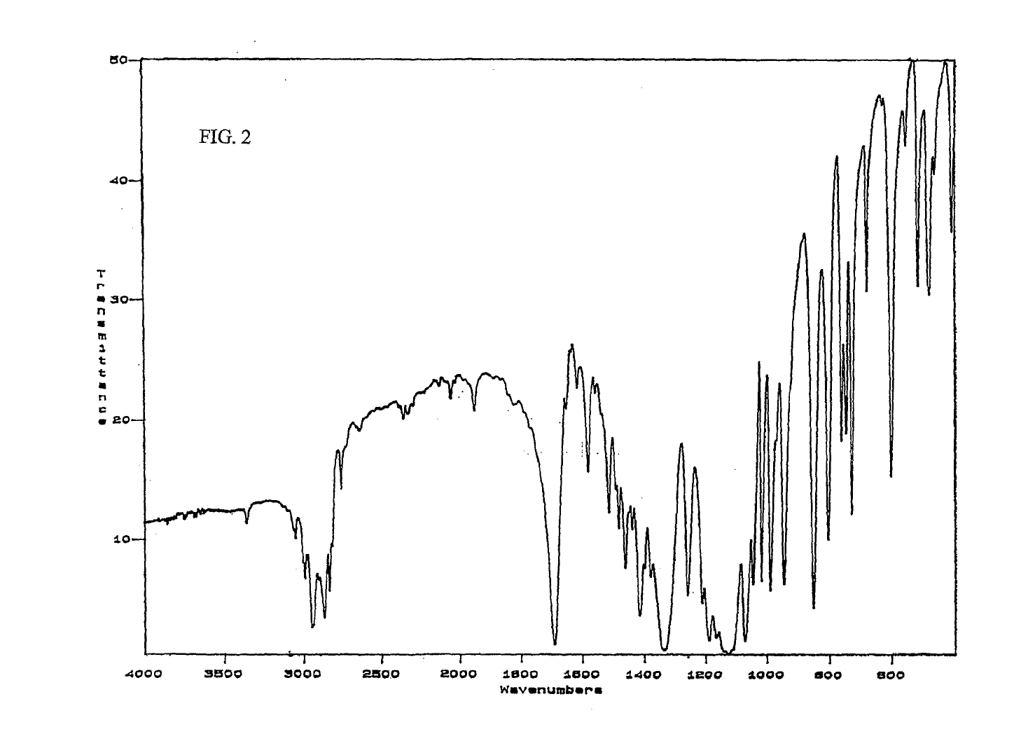

FIG. 3: Analytical Profile of the final product 4-trifluoromethylvalerophenone—mass spectroscopy.

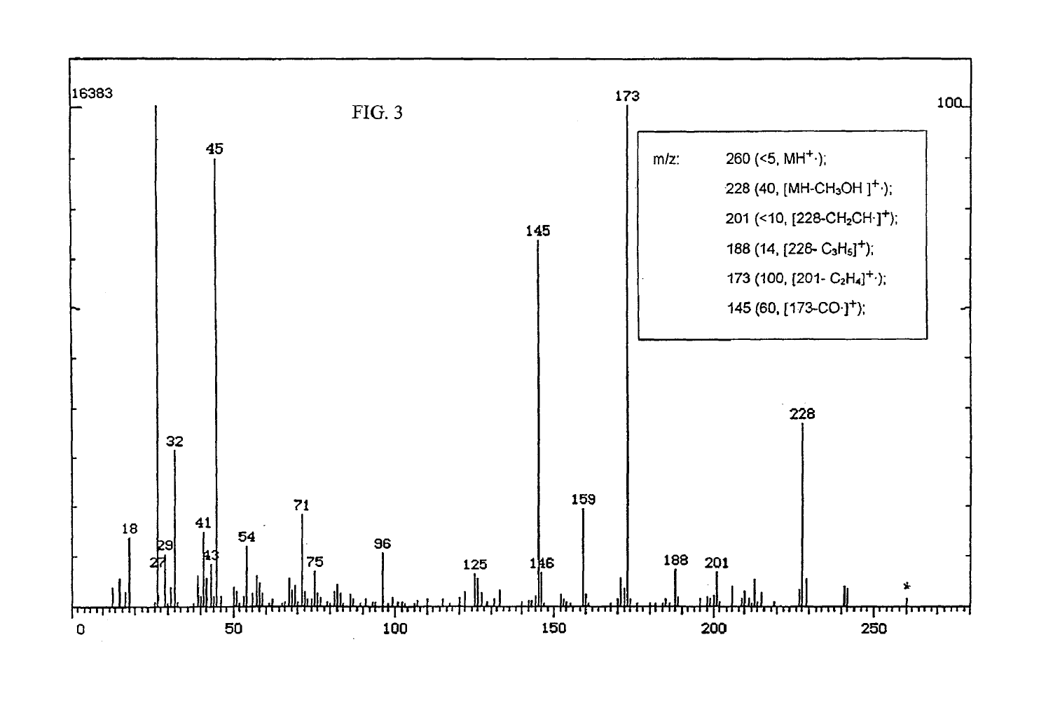

### DETAILED DESCRIPTION OF THE INVENTION

The aims and advantages of the process for the synthesis and purification of 4-trifluoromethylvalerophenone, object of the present invention, will be better understood in the course of the following detailed description. Further implementation or adaptations as well as embodiments readily apparent to those skilled in the art are to be considered within the scope of the present invention.

The present invention provides a process for the synthesis and the purification of trifluoromethylvalerophenone consisting in the steps of:
1. synthesis of organomagnesium compound of 4-halo-methoxybutane
2. coupling of the organomagnesium specie with 4-trifluoromethyl benzonitrile or 4-trifluoromethylbenzoyl chloride
3. purification of the product obtained after coupling.

For the purposes of the present invention the process has to be in particular conducted at the following conditions:
synthesis of organomagnesium specie CH₃O(CH₂)₄MgX, where X is preferably Cl, reacting CH₃O(CH₂)₄X with Mg in condition of reflux in presence of a suitable initiator selected in the group consisting of bromoethane, dibromoethane, bromine, iodine, Vitride® or anthracene in a reaction medium formed by organic solvents selected in the group consisting of 2-methyl-tetrahydrofuran and mixtures thereof with polar aprotic ethereal solvents such as for example tetrahydrofuran, diisopropylether or apolar aprotic non chlorinated solvents such as for example toluene, benzene, xylenes;
synthesis and recovery of 4-trifluoromethylvalerophenone by a coupling reaction obtained in the same reaction medium as in the previous step at temperatures comprised from −5° C.-5° C. by adding or pouring 4-trifluoromethylbenzonitrile or 4-trifluoromethylbenzoyl chloride in the same solvents used for Grignard reaction to CH₃O(CH₂)₄MgX first obtained, treating the mixture reaction with hydrochloric acid diluted solutions, separating the organic layer and concentrating the same to dryness;
purification of 4-trifluoromethylvalerophenone by dissolving the crude product with solvents having boiling point not less than 35° C. to 145° C. selected in the group consisting of aliphatic or aromatic hydrocarbons with C comprised from 2 to 10, lower alkyl alcohol or mixtures of aliphatic or aromatic hydrocarbons: lower alkyl alcohol or mixtures of lower alkyl alcohol:water at temperatures comprised from 20° C. and 145° C. and crystallising the product from the solutions at temperature ranging from −5° C. to 50° C. or conducting on the crude product a batch distillation or thin film distillation at atmospheric pressure or under vacuum.

In one preferred embodiment the reaction medium is formed by 2-methyl-tetrahydrofuran either for the synthesis of organomagnesium specie CH₃O(CH₂)₄MgX and coupling reaction, being the same solvent used for the dissolution of addition 4-trifluoromethylbenzonitrile or 4-trifluoromethylbenzoyl chloride added in this reaction. In an other preferred embodiment the reaction medium is formed by a mixture of 2-methyl-tetrahydrofuran and apolar aprotic solvents, and preferably toluene, either for the synthesis of organomagnesium specie CH₃O(CH₂)₄MgX and coupling reaction.

The 2-methyl-tetrahydrofuran is the preferred solvent having showed the capacity to be suitable for the preparation of CH₃O(CH₂)₄MgX and for separation of the final product being non-miscible with water.

Furthermore the Applicant has found that even mixtures of 2-methyltetrahydrofuran with apolar aprotic solvents such as toluene, benzene, xylenes can be suitable for the Grignard reaction of CH₃O(CH₂)₄MgX preparation in spite of the fact that the latter solvents are not polar solvents and then as known in the art unsuitable for the organomagnesium specie synthesis.

In the preparation of 4-trifluoromethylvalerophenone, the critical point as well known is the preparation of organomagnesium specie being essential at this aim appropriate solvents in presence of suitable initiators.

In conventional industrial methods, the organomagnesium specie preparation can be afforded by reaction of 1-chloro-4-methoxybutane with magnesium in presence of an initiator like iodine, bromine, bromoethane or 1,2-dibromomethane Vitride® or anthracene in polar aprotic solvents, preferably tetrahydrofuran, in order to activate the magnesium turnings. With this meaning, the reaction can be performed with magnesium in its powdered form.

Differently from the known art for the purposes of the present invention the synthesis of organomagnesium specie CH₃O(CH₂)₄MgX is conducted in conditions different from those above mentioned, being the 2-methyltetrahydrofuran essential at the aim to fulfil the purposes of the present invention. In fact with the use of 2-methyltetrahydrofuran the Applicant has found that it is possible to overcome the technical problem due to the use of appropriate solvents suitable as reaction medium both for the organomagnesium specie preparation and for the following coupling reaction with complete recovery of the final product, without the necessity to extract the crude product from the reaction mixture with chlorinated solvents.

Even with similarity in the molecular structure and in some of the physical data resumed in the following table, tetrahydrofuran and methyltetrahydrofuran are significantly different from process chemistry point of view: the first one is for example soluble in all part in water where the second doesn't. That means that methyltetrahydrofuran can be easily keep anhydrous with a simple extraction and conventional dehydrating agents, where tetrahydrofuran need an accurate and time consuming distillation to be rectified in the required anhydrous form to be used in the Grignard preparation.

| | CAS | | | | | |
|---|---|---|---|---|---|---|
| Chemical | Registry | | @P | | | |
| Name | Number | BP ° C. | (torr) | CRT | CP (J mol−1 grd−1) | @T |
| tetrahydrofuran | 109-99-9 | 64-66ⁱ | 760 | 268ⁱⁱ | 76.87-136.5ⁱⁱⁱ | −153.1-−13.1 |
| | | 62-63^{iv} | 720 | | 120.3-128.1^{v} | 10-40 |
| | | 31^{vi} | 208 | | 124.05-127.91^{vii} | 25-40 |
| | | 25^{viii} | 150 | | | |
| 2-methyl- | 96-47-9 | 77-78^{ix} | 716 | 264 | 156.89-161.56^{x} | 25-40 |
| tetrahydro-furan | | | | | | |
| | | 68-70^{xi} | 648 | | | |

^{I}Patent; Shell; BE 632243; 1963; Chem. Abstr.; EN; 61; 644; 1964
^{II}Kobe et al.; J. Chem. Eng. Data; 1; 1956; 50, 53
^{III}Leaist, D. G.; Murray, J. J.; Post, M. L.; Davidson, D. W.; J. Phys. Chem.; EN; 86; 21; 1982; 4175-4178.
^{IV}Klages; Moehler; Chem. Ber.; 81; 1948; 411, 417.
^{V}Costas, Miguel; Patterson, Donald; J. Chem. Soc. Faraday Trans.1; EN; 81; 1985; 2381-2398
^{VI}Kaesz et al.; J. Amer. Chem. Soc.; 82; 1960; 6228, 6231.
^{VII}Rodriguez, S.; Lafuente, C.; Artigas, H.; Royo, F. M.; Urieta, J. S.; J. Chem. Thermodynamics; EN; 31; 1; 1999; 139-150.
^{VIII}Brown, H. C.; Gupta, S. K.; J. Amer. Chem. Soc.; EN; 97; 1975; 5249-5255.
^{IX}Lipp; Chem. Ber.; 22; 1889; 2569
^{XI}Rodriguez, S.; Lafuente, C.; Artigas, H.; Royo, F. M.; Urieta, J. S.; JCTDAF; J. Chem. Thermodynamics; EN; 31; 1; 1999; 139-150
^{XI}Walling, C.; Bristol, D.; J. Org. Chem.; EN; 37; 1972; 3514-3516

In preferred embodiments for the industrial application of the process these solvents are, as mentioned before, the 2-methyltetrahydrofuran and mixture of 2-methyltetrahydrofuran:toluene. In the case of use of mixture of solvents the ratio can be comprised between 0.25-1.0 to 0.25-1.5, being the ratio of 1-1.13 the preferred one. Toluene in equivalent molar mixture with 2-methyltetrahydrofuran (molar ratio with the substrate of the Grignard preparation 0.95-1.0) have less environmental impact and furthermore, due to their immiscibility with water, allow a complete recovery of the coupling product at the end of the reaction without further extraction with chlorinated solvents.

Furthermore even the analytical profile of the product 4-chloromethoxybutane in the activation phase during the Grignard synthesis is particularly relevant in the organomagnesium preparation, together with the characteristics of magnesium and the type of initiator.

As for the reacting product 1-chloro-4-methoxybutane, Hara and co-workers first described its preparation in an affordable way by reaction of tetrahydrofuran and methylchlorosulfinates (J. Organic Chem., 1975, 40, 2786-2791). This was a remarkable achievement according to the authors being with ethylene oxide the chloroethylmethylsulfite generally the main product.

For the purpose of the present invention, the synthesis of organomagnesium compound is strictly related to the quality of the staring material, so that 1-chloro-4-methoxybutane needs to be free of impurities which can be responsible for inhibition on Grignard synthesis. In particular to avoid the presence of the typical impurity dimethylsulfite of the known method of synthesis, the 1-chloro-4-methoxybutane has to be carefully purified as example by distillation in order to obtain less than 0.5% level of dimethylsulfite impurity. The known conditions to purify the product by distillation under reduced pressure and temperature conditions at 50° C. and 40 torr could not be sufficient to guarantee the desired purity in the scale-up of the process, so a batch distillation or better thin film distillation at 140-150° C. under atmospheric pressure are preferred.

With reference to initiators the preferred at the aim of the present invention is bromoethane, being a friendly-environment initiator, while the magnesium has to be in turning of suitable purity, size and surface, usually with apparent density ranging from 0.4-0.9 g/cm³, preferably from 0.55 to 0.7.

At the reaction mixture 4-trifluoromethyl-benzonitrile in the same organic solvents, employed for the first step, and preferably 2-methyltetrahydrofuran or toluene, is added or poured for the coupling reaction. The same is applied when is used 4-trifluoromethylbenzoyl chloride instead of 4-trifluoromethyl-benzonitrile.

The crude product obtained after reaction coupling is a yellowish to brownish oil, which solidify after cooling a having a overall chromatographic purity between 80-90% so that an additional purification step has to be preferably performed in order to have a 4-trifluoromethylvalerophenone suitable for a preparation of an active ingredients of a pharmaceutical purity grade. For this step a preferred embodiment of the present invention is dissolving the crude product with aliphatic or aromatic hydrocarbons with C comprised from 2 to 10, lower alkyl alcohol or a mixture of said hydrocarbons: said lower alkyl alcohol or a mixture of said lower alkyl alcohol:water at temperatures comprised from 20° C. and 145° C. and preferably at temperatures of 50° C. and then separate the purified product by crystallisation at temperatures in a range from −5° C. to 50° C. The aliphatic or aromatic hydrocarbons with C comprised from 2 to 10 selected in the group consisting of ligroin, cyclohexane, n-heptane, heptanes, n-hexane, hexanes, toluene, petroleum ether and preferably ligroin or petroleum ether, cyclohexane or n-heptane and heptanes. The lower alkyl alcohols can be selected in the group formed by methanol, ethanol, isopropanol, n-butanol, sec-butanol, isobutanol, amyl alcohol and isoamyl alcohol and preferably methanol, ethanol or isopropanol.

The preferred mixtures aliphatic or aromatic hydrocarbons: lower alkyl alcohols are with petroleum ether or ligroin, heptanes, cyclohexane.

The most preferred embodiment for the purpose of the present invention is dissolving with methanol:water in a ratio between 3:1 at 40° C.

Alternatively the purification can be performed by batch distillation or thin film distillation at atmospheric pressure or under vacuum from the crude product.

In the following table the results of purification obtained with dissolutions with different aliphatic or aromatic hydrocarbons, alcohols or mixtures in comparison with purification by distillation and temperatures of crystallisation are reported.

TABLE 1
assessment on the purification of 5-Methoxy-1-(4-trifluoromethyl1-
phenyl1)-pentan-1-one

| Entry | Volumes¹ | Solvents² | | T° C.³ | Yeld⁴ | Purity sm⁵ | Purity ip⁶ |
|---|---|---|---|---|---|---|---|
| 1 | 2 | Ethanol | — | 30 | 71.6 | 94.6 | 99.3 |
| 2 | 2.12 | Ethanol | — | 30 | 68.0 | 87.3 | 99.1 |
| 3 | 1 | Isobutanol | — | 40 | 40.0 | 87.3 | 97.7 |
| 4 | 1 | Ethanol | — | 40 | 85.0 | 99.0 | 99.9 |
| 5 | 1 | Isopropanol | — | 38 | 32.0 | 87.3 | 99.6 |
| 6 | 1 | n-butanol | — | 38 | 50.0 | 87.3 | 99.2 |
| 7 | 1 | Toluene | — | 39 | 23.0 | 87.3 | 99.5 |
| 8 | 1 | Cyclohexane | — | 40 | 74.5 | 87.3 | 99.2 |
| 9 | 1 | Isobutanol | — | 39 | 73.0 | 87.3 | 98.9 |
| 10 | 1 | Isobutanol | Water (5) | 39 | 49.5 | 87.3 | 99.0 |
| 11′ | 1 | Ethanol | — | 40 | 59.1 | 93.0 | 99.0 |
| 11 | 1 | Methanol | Water (10) | 38 | 53.0 | 87.3 | 99.8 |
| 12 | 1 | Ethanol | Water (10) | 38 | 22.0 | 87.3 | 98.9 |
| 13 | 1 | Methanol | Water (30) | 41 | 76.0 | 87.3 | 95.2 |
| 14 | 1 | Hexanes | — | 40 | 81.5 | 87.3 | 97.4 |
| 15 | 1 | Petroleum | — | 40 | 79.0 | 87.3 | 98.7 |
| | | ether | | | | | |
| 16 | 2 | Isobutanol | — | 40 | 70.6 | 87.3 | 99.6 |
| 17 | 2 | Petroleum | — | 40 | 76.0 | 87.3 | 98.6 |
| | | ether | | | | | |
| 18 | 3 | Isobutanol | Water (66) | 41 | 86.0 | 87.3 | 98.7 |
| 19 | 2 | Isobutanol | Water (75) | 41 | 96.0 | 87.3 | 97.8 |
| 20 | 2 | Isobutanol | HCl_{aq} 5% | 42 | 88.0 | 87.3 | 98.7 |
| | | | (75) | | | | |
| 21 | 2.5 | Isobutanol | Water (60) | 41 | 88.0 | 84.0 | 97.5 |
| 22 | 2.5 | Methanol | Water (17) | 30 | 80.0 | 84.0 | 97.1 |
| 23 | 3 | Methanol | Water (30) | 32 | 98.5 | 84.0 | 95.5 |
| 24 | 3.2 | Heptanes | Isopropanol | 25 | 37.0 | 84.0 | 99.6 |
| | | | (6) | | | | |
| 25 | 3.2 | Methanol | Water | 30 | 100 | 84.0 | 97.8 |
| | | | (31)- | | | | |
| | | | toluene (7) | | | | |
| 26 | 2.75 | Methanol | Water (27) | 30 | 94.0 | 84.0 | 98.1 |
| 27 | 1.7 | Heptanes | Isopropanol | 30 | 50.0 | 84.0 | 99.5 |
| | | | (12) | | | | |
| 28 | 2.2 | Heptanes | Isopropanol | 20 | 70.0 | 84.0 | 98.9 |
| | | | (9) | | | | |
| 29 | 2.75 | Methanol | Water (27) | 24 | 86.0 | 84.0 | 96.1 |

| | | | | |
|---|---|---|---|---|
| 30 | distillation | 76.0 | 88.3 | 99 |

¹Volume of solvent mixture per parts of substrates (litre per Kg)
²Nature of solvents used. Under brackets percentage by weight of the solvent in the mixture, is indicated.
³Maximum temperature at which the crystallization has been carried out our germination of crystals was observed
⁴Yield refer on the isolated product
⁵Chromatographic purity on the starting material
⁶Chromatographic purity on the isolated product.

The following examples are given at illustrative and not limiting purpose of the invention.

### Example 1

### Preparation of 1-chloro-4 Methoxybutane

In a glass-lined reactor of suitable size, a solution of 65.68 kg (553 moles) of thionyl chloride is charged. Maintaining the temperature between 10-20° C. and anyway not exciding the 25° C., a solution of 15.4 Kg (481 moles) of methanol, is slowly added. To that mixture at 20-25° C. a solution of 34.6 kg (480 moles) of tetrahydrofuran is added. After the completion of the addition, the mixture is slowly heated until reflux and maintained at reflux for 4-5 hours. The constant reaction of the substrates to the conversion of the desired product, is observed on the continuos, quasi linear grow of the boiling point of the reaction mixture from the original 68-72° C. to the final 119° C. After this time the reaction mixture is cooled to 20-25° C. in around 30-45 minutes. The reaction mixture is quenched 120 litres of water in 5 portions until neutral pH; the organic phases are collected together. 50.8 kg of crude product are obtained. The crude product is then distilled at atmospheric pressure at 140-150° C., collecting 22 kg of pure product (purity Area Product by Gas-Chromatography 97-98% of which dimethylsulphite below 0.5%; molar yield: 37-38%).

### Example 2

### Preparation of 4-methoxybutan-1-magnesium Chloride

In a suitable reactor 1.8 kg (74.0 moles) of magnesium are suspended in 7.2 L of 2-methyltetrahydrofuran (6.2 kg; 72.0 moles) under stirring and inert atmosphere (nitrogen). The Grignard reaction is initiate with 7.5 g of bromoethane at 45-50° C., preferably between 47-49° C. Then the solution of 9.0 kg of 1-chloro-4-methoxybutane (73.4 moles) in 5.6 L (4.8 kg) of toluene, is added under stirring in 3-5 hours, dosing the rate of addition in a way to keep the mixture at reflux. The reaction mixture is heated under reflux for further 60 minutes.

After this phase the reaction mixture is cooled at 45-50° C. and 20 L (17.3 kg) of toluene are added. The reaction mixture is used in the next step without further isolation or purification.

### Example 3

### Preparation of 5-methoxy-1-(4-trifluoromethyl-phenyl)-pentan-1-one: Coupling Step

To the previous reaction mixture cooled between −2÷−8° C., a solution of 4-trifluoromethyl-benzonitrile 7.5 kg (43.8 moles) in 14 L of toluene are added in 4-6 hours, preferably from 4.5 to 5.5 hours. The rate of addition is adjust to keep the temperature below 0° C. At the end the reaction mixture is kept under stirring additionally for 4 hours more at −1÷+1° C.

Subsequently, the reaction mixture is quenched on aqueous solution of hydrochloric acid 10% w/w.

The aqueous organic mixture is diluted additionally with further 5 L of toluene, checking the pH at value comprise between 1÷2. The mixture is heated at 40-45° C. and the organic phase is separated from the aqueous one. The organic phase is then washed three times with water and concentrated until dryness. The yellowish-brownish oil (around 9 kg) of the title compound confirmed by spectroscopic attribution, is purified in the following step.

### Example 4

### Preparation of 5-methoxy-1-(4-trifluoromethyl-phenyl)-pentan-1-one: Coupling Step

The reaction mixture coming from the example 2 cooled between −2÷−8° C., a solution of 4-trifluoromethyl-benzonitrile 2.5 kg (14.6 moles) in 4.5 L of 2-Methyltetrahydrofuran are added in 4-6 hours, preferably from 4.5 to 5.5 hours. The rate of addition is adjust to keep the temperature below 0° C. At the end the reaction mixture is kept under stirring additionally for 4 hours more at −1÷+1° C.

Subsequently, the reaction mixture is quenched on aqueous solution of hydrochloric acid 10% w/w.

The aqueous organic mixture is diluted additionally with further 2 L of 2-Methyltetrahydrofuran, checking the pH at value comprise between 1÷2. The mixture is heated at 40-45° C. and the organic phase is separated from the aqueous one. The organic phase is then washed three times with water and concentrated until dryness. The yellowish-brownish oil (around 9 kg) of the title compound confirmed by spectroscopic attribution, is purified in the following step.

### Example 5

### Purification of 5-Methoxy-1-(4-trifluoromethyl-1-phenyl1)-pentan-1-one

The oil of the crude product from the previous step is diluted with 22.5 L of methanol. The mixture is heated at 50-55° C. and then until reflux which is maintained for 20-40 minutes. The solution is then cooled between 10 to 20° C., preferably at 14-16° C. and 7.5 L of water in 3-5 hours, are added. The suspension is stirred at this temperature for 2 hours and then filtered, washing the cake with water 8.5 g of desired product are obtained with spectroscopic data according to the structure required, purity by HPLC in Area Product, not less than 99% and moisture contents from 10 to 15% (molar yield: 63/67% on the dry product).

### NMR and MS Analytical Characterisation of the Final Product

¹H-NMR (CDCl₃; 200 MHz): 8.03 (2H; d; J: 8.2; 3′, 5′-H); 7.69 (2H; d; J: 8.2; 2′, 6′-H); 3.40 (2H; t; J: 6.2; 2-CH₂); 3.30 (3H; s; —OCH₃); 3.01 (2H; t; J: 6.9; 5-CH₂); from 1.90 to 1.57 (4H; m; 3,4-CH₂);

MS: 260 (<5, MH⁺); 228 (40, [MH−CH₃OH]⁺); 201 (<10, [228-CH₃OH]⁺); 188 (14, [228-C₃H₅]⁺); 173 (100, [201-C₂H₄]⁺); 145 (60, [173-CO⁻]⁺)

1-16. (canceled)

17. Process for the synthesis of (4-methoxybutyl)(4-trifluoromethylphenyl)methanone comprising:
synthesising an organomagnesium specie CH₃O(CH₂)₄MgX, where X is an halogen, reacting CH₃O(CH₂)₄X with Mg in presence of a suitable initiator in a reaction medium formed by 2-methyl-tetrahydrofuran in admixture with organic solvents selected in the group consisting of polar aprotic ethereal solvents or apolar aprotic non chlorinated solvents; and
synthesising and recovering said (4-methoxybutyl)(4-trifluoromethylphenyl)methanone by a coupling reaction of CH₃O(CH₂)₄MgX by adding in the reaction mixture first obtained 4-trifluoromethylbenzonitrile or 4-trifluoromethylbenzoyl chloride dissolved in the same organic solvents of the first step, treating the mixture reaction obtained with aqueous acidic diluted solutions, separating the organic layer, and concentrating the same to dryness.

18. Process for the synthesis of (4-methoxybutyl)(4-trifluoromethylphenyl)methanone according to claim 17, further comprising purifying said (4-methoxybutyl)(4-trifluoromethylphenyl)methanone by distillation or crystallisation from solutions of the crude product obtained with solvents having boiling point not less than 35° C. and below 145° C.

19. Process for the synthesis of (4-methoxybutyl)(4-trifluoromethylphenyl)methanone according to claim 17, wherein the 2-methyl-tetrahydrofuran is in a molar ratio with the substrate of the Grignard preparation of 0.95-1.0.

20. Process for the synthesis of (4-methoxybutyl)(4-trifluoromethylphenyl)methanone according to claim 17, wherein the reaction medium is formed by mixtures of 2-methyl-tetrahydrofuran with polar aprotic ethereal solvents selected in the group consisting of tetrahydrofuran, diisopropylether.

21. Process for the synthesis of (4-methoxybutyl)(4-trifluoromethylphenyl)methanone according to claim 17, wherein the reaction medium is formed by a mixture of 2-methyl-tetrahydrofuran with apolar aprotic non chlorinated solvents, said solvent is at least one selected from the group consisting of: toluene, benzene, and xylene.

22. Process for the synthesis of (4-methoxybutyl)(4-trifluoromethylphenyl)methanone according to claim 21, wherein the reaction medium is formed by equimolar mixtures of 2-methyl-tetrahydrofuran:toluene.

23. Process for the synthesis of (4-methoxybutyl)(4-trifluoromethylphenyl)methanone according to claim 17, wherein the Grignard reaction of the first step is conducted in reflux conditions.

24. Process for the synthesis of (4-methoxybutyl)(4-trifluoromethylphenyl)methanone according to claim 17, wherein CH₃O(CH₂)₄X has less than 0.5% impurities.

25. Process for the synthesis of (4-methoxybutyl)(4-trifluoromethylphenyl)methanone according to claim 17, wherein X is chlorine.

26. Process for the synthesis of (4-methoxybutyl)(4-trifluoromethylphenyl)methanone according to claim 17, wherein the initiator for organomagnesium specie CH₃O(CH₂)₄MgX preparation is at least one selected from the group consisting of: bromoethane, dibromoethane, bromine, iodine, Vitride® and anthracene.

27. Process for the synthesis of (4-methoxybutyl)(4-trifluoromethylphenyl)methanone according to claim 26, wherein the initiator for organomagnesium specie CH₃O(CH₂)₄MgX preparation is bromoethane.

28. Process for the synthesis of (4-methoxybutyl)(4-trifluoromethylphenyl)methanone according to claim 17, wherein the magnesium has an apparent density ranging from 0.4-0.9 g/cm³.

29. Process for the synthesis of (4-methoxybutyl)(4-trifluoromethylphenyl)methanone according to claim 18, wherein the magnesium has an apparent density ranging from 0.55 to 0.7 g/cm³.

30. Process for the synthesis of (4-methoxybutyl)(4-trifluoromethylphenyl)methanone according to claim 17, wherein the coupling reaction is conducted at a temperature in the range between −5° C. and 5° C.

31. Process for the synthesis of (4-methoxybutyl)(4-trifluoromethylphenyl)methanone according to claim 18, wherein the crystallisation of the purification step is conducted in at least one solvent selected from the group consisting of: C₂ to C₁₀ aliphatic or aromatic hydrocarbons, C₁ to C₅ lower alkyl alcohols, mixtures of said aliphatic or aromatic hydrocarbons and said lower alkyl alcohols, and mixtures of said lower alkyl alcohols and water at temperatures in the range between 20° C. to 145° C. and said crystallisation occurring at temperature in the range between −5° C. and 50° C.

32. Process for the synthesis of (4-methoxybutyl)(4-trifluoromethylphenyl)methanone according to claim 31, wherein said solvent is at temperature of 50° C.

33. Process for the synthesis of (4-methoxybutyl)(4-trifluoromethylphenyl)methanone according to claim 31, wherein said solvent at least one selected from the group consisting of: ligroin or petroleum ether, cyclohexane, n-heptane, heptanes, n-hexane, hexanes, toluene, methanol, ethanol, isopropanol, n-butanol, sec-butanol, isobutanol, amyl alcohol and isoamyl alcohol.

34. Process for the synthesis of (4-methoxybutyl)(4-trifluoromethylphenyl)methanone according to claim 31, wherein the aliphatic or aromatic hydrocarbons solvent in the mixture of aliphatic or aromatic hydrocarbon and lower alkyl alcohol is at least one aliphatic or aromatic hydrocarbon solvent selected from the group consisting of: petroleum ether or ligroin, heptanes, and cyclohexane.

35. Process for the synthesis of (4-methoxybutyl)(4-trifluoromethylphenyl)methanone according to claim 31, wherein the purification is conducted with a solvent comprising a mixture of methanol and water in a ratio of 3:1 at 40° C.

36. Process for the synthesis of (4-methoxybutyl)(4-trifluoromethylphenyl)methanone according to claim 18, wherein the purification step is conducted by batch distillation or thin film distillation at atmospheric pressure or under vacuum.
